(19) United States
(12) Patent Application Publication
(10) Pub. No.: US 2002/0128682 A1
(43) Pub. Date: Sep. 12, 2002

(54) Surgical forceps

(75) Inventors: Stephan Prestel, Rheinstetten-Morsch (DE); Daniel Dietzel, Bretten- Diedelsheim (DE); Helmut Heckele, Knittlingen (DE); Ernst Falk, Sternenfels-Diefenbach (DE)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: Richard Wolf GmbH

(21) Appl. No.: 10/094,084

(22) Filed: Mar. 8, 2002

(30) Foreign Application Priority Data
Mar. 9, 2001 (DE) 101 11 510.5

(51) Int. Cl. A61B 17/44

(52) U.S. Cl. 606/205

(57) ABSTRACT

The invention relates to a surgical forceps with a shank and with a distal-side forceps jaw with two jaw parts which are sharp-cutting on closing the forceps jaw and of which the first jaw part is pivotable relative to the other stationary second jaw part by way of axial adjustment of an actuation element. For improving the handling according to the invention it is envisaged that the two jaw parts which in each case are formed two-limbed, in the longitudinal section are configured essentially L-shaped.

### BACKGROUND OF THE INVENTION

The invention relates to a surgical forceps with a shank and with a distal-side forceps jaw with two jaw parts which are sharp-cutting on closing the forceps jaw and of which the first jaw part is pivotable by way of axial adjustment of an actuation element relative to the other stationary second jaw part.

The ethmoid roof of humans from a point of view with regard to operation technology makes particular demands on instrumentation. In order in this region of the nasal cavities to be able to separate tissue without the risk for example of a cerebrospinal fluid fistula there are required instruments whose shaping is adapted to the particular anatomy of the nose cavity.

In the state of the art there are known a multitude of instruments for surgery of the sinus. In particular there exist ethmoid forceps which however only permit the lateral working, but not the working on the roof of the organ system. Special forceps or punches although permitting the operation on the roof of the organ system, they however have various disadvantages. On account of their solid design the forceps or punches can only be led to the region of operation with great difficulty. Furthermore this also causes a considerable limitation of view through the instrument which makes it quite difficult for the operator to have an overview of the zone of the cavity region to be treated. Furthermore it has been shown to be disadvantageous that previously known ethmoid forceps tend to tear off tissue which increases the risk of a cerebrospinal fluid fistula.

### BRIEF SUMMARY OF THE INVENTION

It is therefore the object of the invention to provide a surgical forceps of the previously mentioned type which no longer has the mentioned disadvantages. It is therefore to be suitable to be able to carry out operations in the roof region of the nose organ system. Finally a tear-free cutting of tissue is to be ensured.

The solution of this object by way of the invention is characterized in that the two jaw parts which in each case are formed two-limbed, in the longituudinal section are configured essentially L-shaped. This shape is usefully achieved such that with a closed forceps jaw the one limb of each jaw part arc runs inclined at an angle to the longitudinal axis of the shank and via a jaw part arc blends into the other limb which is bent at a substantial angle towards the side. The shank is advantageously formed tubular, wherein the actuation element runs in the inside of the shank.

A particularly good course of the cut through the tissue results when the angle enclosed by the two limbs of each jaw part is larger than 90°. For the secure gripping of the tissue on the cutters of the forceps it is furthermore envisaged that on one of the two jaw parts, preferably on the stationary jaw part, bordering its cutter and parallel to this there is provided a pronounced profile. With this it may be the case of a grooving or a serration.

The handling ability of the forceps is much more comprehensive when the forceps insert consisting of the shank, actuation element and forceps jaw are rotatable and fixable with respect to the handle of the forceps.

With the design according to the invention it becomes possible not only to carry out a cut in the longitudinal direction of the instrument but also transverse to the longitudinal axis of the instrument. Furthermore the region in the body cavity to undergo therapy may be simply reached. The suggested forceps may, as has already been mentioned, carry out a sharp cut in the direction of the instrument longitudinal axis as well as a sharp cut running transverse to this over a certain length, so that one advantage of the suggested forceps also lies in the fact that there exists the possibility of a lateral leading of a cut, in particular as good leading of the cut with the operation on the roof of the organ system.

### BRIEF DESCRIPTION OF THE DRAWINGS

In the drawing there is represented one embodiment example of the invention. There are shown in

FIG. 1 a surgical forceps in the lateral view,

FIG. 2 the distal end section of the forceps in an enlargement, with a closed forceps jaw,

FIG. 3 the end section shown in FIG. 2, in a plan view and

FIG. 4 the distal end section of the forceps according to FIG. 2 with an opened forceps jaw.

### DETAILED DESCRIPTION OF THE INVENTION

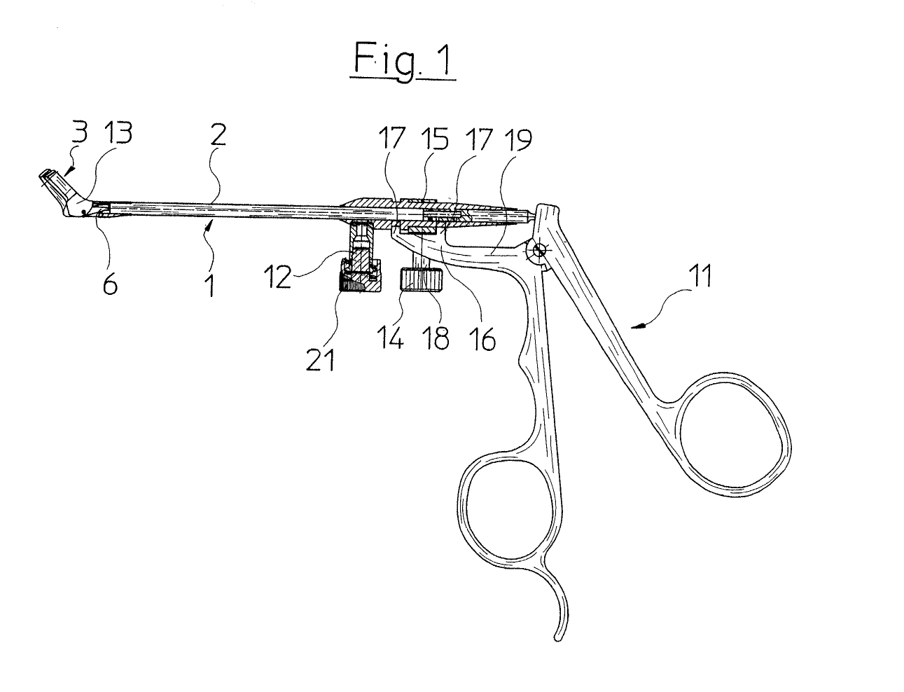

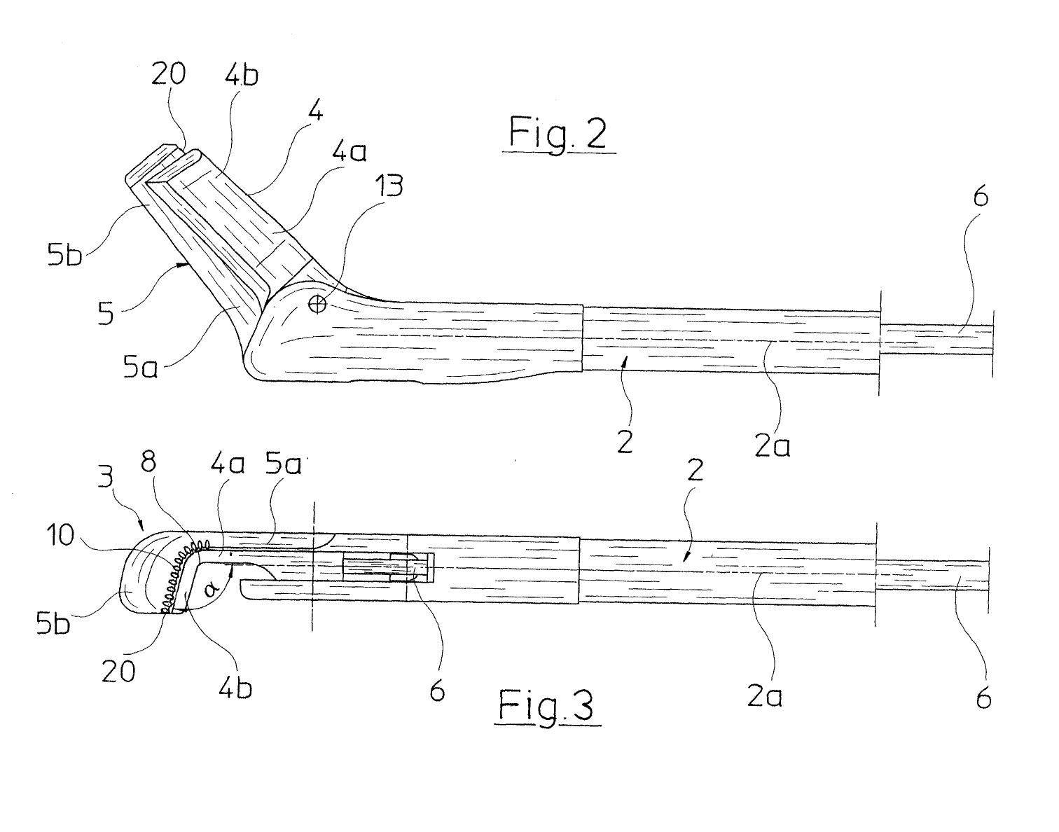

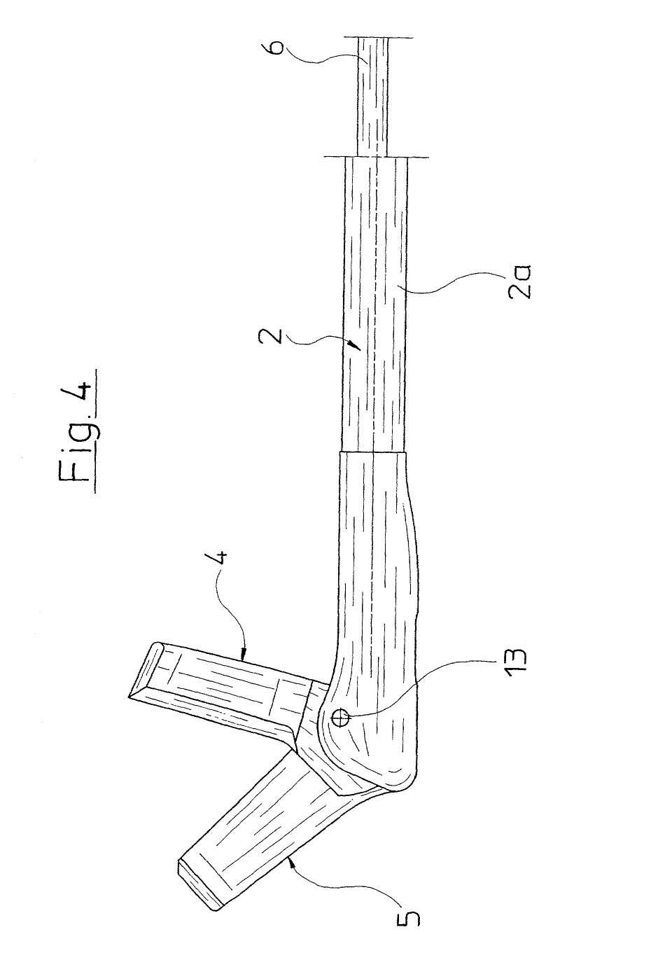

In FIG. 1 there is to be seen laterally in the lateral view a surgical forceps 1 which in particular is suitable for the operation of the ethmoid. The forceps 1 has a shank 2 which connects the forceps jaw 3 and the handle 11 or the forceps housing to one another. The forceps jaw 3 consists of two jaw parts 4 and 5. One jaw part 5 at the same time is rigidly arranged on the shank 2 whilst the other jaw part 4 by way of a joint pin 13 is pivotably mounted in a movable manner and relative to the jaw part 5. By way of the pivoting movement on closing the forceps jaw tissue may be severed. The jaw parts provided with cutters at the same time act as scissors.

The pivoting of the movably arranged jaw part 4 with respect to the rigid jaw part 5 is effected for closing or opening the forceps jaw by way of an axial displacement movement of an actuation element 6 formed as a rod, which is guided concentrically in the tubularly formed shank 2. The axial movement of the actuation element 6 is effected in a manner known per se by way of a suitable actuation of the handle 11.

Both jaw parts 4 and 5 consist, as is best deduced from FIG. 3, of two limbs 4a, 4b and 5a, 5b which via a jaw part arc 8 blend into one another. The one limb 4a or 5a of each jaw part 4 and 5 runs with a closed jaw part 3 inclined at an angle to the longitudinal axis 2 of the shank 2. The angle between the shank longitudinal axis 2a and limb direction may be about 135°.

In the perpendicular direction to this according to FIG. 3 it is to be recognized that the other limbs 4b, 5b of the two jaw parts 4 and 5 respectively are arranged at an angle to the limbs 4a, 5a. Via a jaw part arc 8 the one limb blends into the limb bent at right angles to the side so that the jaw part in the longitudinal section is configured essentially L-shaped or hook-shaped. The angle a enclosed by the jaw part limbs of each jaw part is preferably more than 90° so that the cut from the beginning to the end along the cutting edges of the limbs is effected advancing in a point-like manner. Thus there is achieved a clean cut since in the region of the limbs 4b, 5b bent at right angles with a small angle otherwise there may result problems in the context of a uniform and complete cut. According to the respective application purpose the distal-side bending at right angles of the two jaw parts 4, 5 towards the side may be effected to the left and to the right.

A considerable improvement of the handling ability of the forceps 1 may be achieved in that the forceps insert, consisting of the shank 2, actuation element 6 and jaw parts 4 and 5 are rotatable and fixable with respect to the handle 11 of the forceps. At the same time the rotation may be effcted over 360°. In order to be able to carry out this rotation the thread sleeve 14 shown in FIG. 1 must be released so that the non-positive connection between the forceps insert and a first annular receiver 15 on the one hand and a fork-shaped second receiver 16 on the other hand may be lifted. As can be deduced from FIG. 1 the annular receiver 15 between two projections 17 is releasably fixed in that the thread stems 18 of the receiver 15 via a bore located in the handle part 19 is engaged with the threaded sleeve 14.

A sliding of the tissue to be severed on and out of the forceps jaw is effectively prevented in that on the stationary jaw part 5 bordering its cutter 20 and parallel to this there is provided a pronounced profile 10 (FIG. 3). The profile 10 here consists of a grooving or of a serration and firmly holds the tissue with the cutting procedure.

For the purpose of cleaning and/or disinfection the shank 2 may be completely released from the handle 11. For rinsing there is provided a rinsing connection 2 which may be closed by way of a cap 21 which may be screwed on. On the rinsing connection 12 rinsing fluid may be introduced into the forceps for rinsing through the inner space of the shank 2 accommodating the actuation element 6.

The actuation element 6 may be sealed with respect to the shank 2 in the region of the proximal-side end by way of an O-ring which is not shown. By this it is effected that the rinsing through of the forceps shank 2 is only effected in the distal direction in order also to alleviate contamination possible also in the region of the two forceps jaw parts 4, 5 and impurities which have penetrated into the shank.

1. A surgical forceps with a shank and with a distal-side forceps jaw with two jaw parts which are sharp-cutting on closing the forceps jaw and of which the first jaw part is pivotable relative to the other stationary second jaw part by way of axial adjustment of an actuation element, wherein the two jaw parts which in each case are formed two-limbed, in the longituudinal section are configured essentially L-shaped.

2. A forceps according to claim 1, wherein with a closed forceps jaw the one limb of each jaw part runs inclined at an angle to the longitduinal axis of the shank and via a jaw part arc blends into the other limb which is bent at a substantial angle towards the side.

3. A forceps according to claim 1 or 2, wherein the shank is designed tubular and the actuation element runs in the inside of the shank.

4. A forceps according to one of the claims 1 to 3, wherein the angle α enclosed by the two limbs (4a, 4b; 5a, 5b) of each jaw part (4,5) is larger than 90°.

5. A forceps according to one of the claims 1 to 4, wherein on one of the jaw parts bordering its cutter and parallel to this there is provided a pronounced profile.

6. A forceps according to claim 5, wherein the profile is provided on the stationary jaw part.

7. A forceps according to claim 5 or 6, wherein the profile is a grooving or a serration.

8. A forceps according to one of the claims 1 to 7, wherein a forceps insert, consisting of shank, actuation element and jaw parts is rotatable and fixable with respect to a handle of the forceps.

9. A forceps according to one of the claims 1 to 8, wherein the jaw part on closing acts as a scissors.
